(19) United States
(12) Patent Application Publication
(10) Pub. No.: US 2020/0353218 A1
(43) Pub. Date: Nov. 12, 2020

(54) Stabilization Device for Invasive Hemodynamic Monitoring Transducer

(71) Applicant: Benjamin-Fausto S. Thielman, Fredericksburg, VA (US)

(72) Inventor: Benjamin-Fausto S. Thielman, Fredericksburg, VA (US)

(21) Appl. No.: 16/406,934

(22) Filed: May 8, 2019

(51) Int. Cl. A61M 25/02

(52) U.S. Cl. CPC A61M 25/02; A61M 2025/024; A61M 2025/0213; A61M 2025/0266; A61M 2025/0253; A61M 2025/0293

(57) ABSTRACT

A stabilization device is provided for attaching an instrument along a phlebostatic axis of a patient's torso, said instrument having a tether to a monitor. The stabilization device includes a flexible pad, an instrument holder, and a protection cover. The flexible pad contacts the torso along the phlebostatic axis. The instrument holder is disposed on the pad. The holder includes a clamp to secure the instrument. The protection cover connects to the holder by a hinge and rotates along the hinge between an open position and a close position. In the close position, the cover contains the clamp and the instrument. A swivel joint connects the holder to the pad for angular independence.

### BACKGROUND

The invention relates generally to medical stabilization device mechanisms. In particular, instruments for monitoring pressure in a patient necessitate maintaining both at the same hydrostatic level.

StatLock° represents a commonly used stabilization device that employs clamps for attaching medical devices to patients. These devices are typically used to secure Foley® catheters to the upper thigh of patients.

### SUMMARY

Conventional stabilization devices yield disadvantages addressed by various exemplary embodiments of the present invention. In particular, exemplary embodiments provide a stabilization device for attaching an instrument along a phlebostatic axis of a patient's torso, said instrument having a tether to a monitor. The exemplary stabilization device includes a flexible pad, an instrument holder, and a protection cover.

The flexible pad contacts the torso along the phlebostatic axis. The instrument holder is disposed on the pad. The holder includes a clamp to secure the instrument. The protection cover connects to the holder by a hinge and pivots along the hinge between an open position and a close position. In the close position, the cover encloses the clamp and the instrument. Additional embodiments include a swivel joint to rotatably connect the holder to the pad.

### BRIEF DESCRIPTION OF THE DRAWINGS

These and various other features and aspects of various exemplary embodiments will be readily understood with reference to the following detailed description taken in conjunction with the accompanying drawings, in which like or similar numbers are used throughout, and in which:

FIG. 1 is an elevation view of a patient with an exemplary stabilization device at the phlebostatic axis;

FIG. 2 is a plan view of the exemplary stabilization device reverse side;

FIG. 3 is a set of plan and elevation views of the stabilization device including its obverse side with an open cover;

FIG. 4 is a set of plan and elevation views an elevation view of the stabilization device with the cover closed; and

FIG. 5 is an isometric view of the pad and holder with a detail cross-section view of swivel components.

### DETAILED DESCRIPTION

In the following detailed description of exemplary embodiments of the invention, reference is made to the accompanying drawings that form a part hereof, and in which is shown by way of illustration specific exemplary embodiments in which the invention may be practiced. These embodiments are described in sufficient detail to enable those skilled in the art to practice the invention. Other embodiments may be utilized, and logical, mechanical, and other changes may be made without departing from the spirit or scope of the present invention. The following detailed description is, therefore, not to be taken in a limiting sense, and the scope of the present invention is defined only by the appended claims.

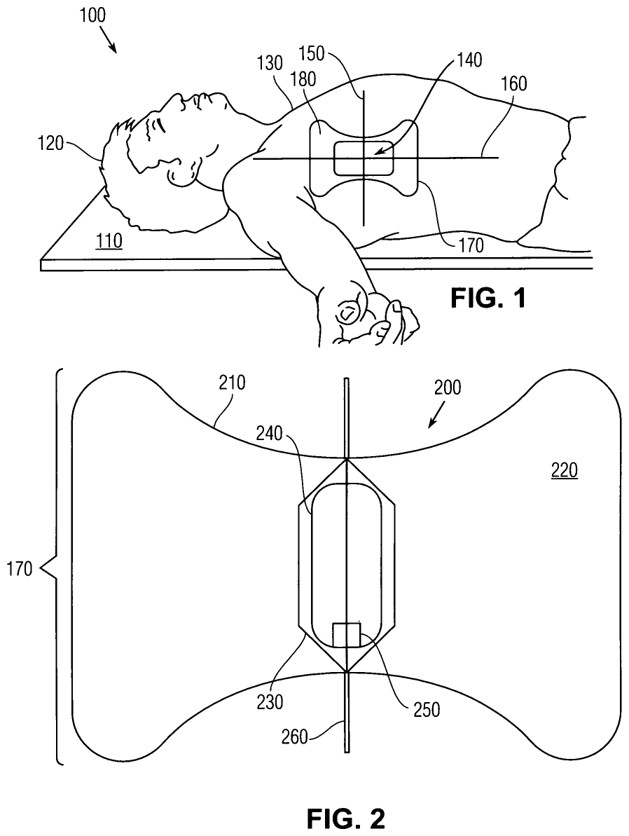

FIG. 1 shows an elevation view 100 of a bed 110 on which a patient's head 120 and torso 130 lie with the phlebostatic axis 140 denoted, which crosses the right atrium of the patient's heart. A sternal line 150 identifies the lateral position of the sternum, while a midchest line 160 identifies a level corresponding to the air zero port of a transducer for monitoring pressure. The phlebostatic axis 140 marks the intersection of the sternal and midchest lines 150 and 160. An exemplary stabilization device or stabilizer 170 attaches to the torso 130 covering the phlebostatic axis 140 with the obverse side 180 facing outward from the patient.

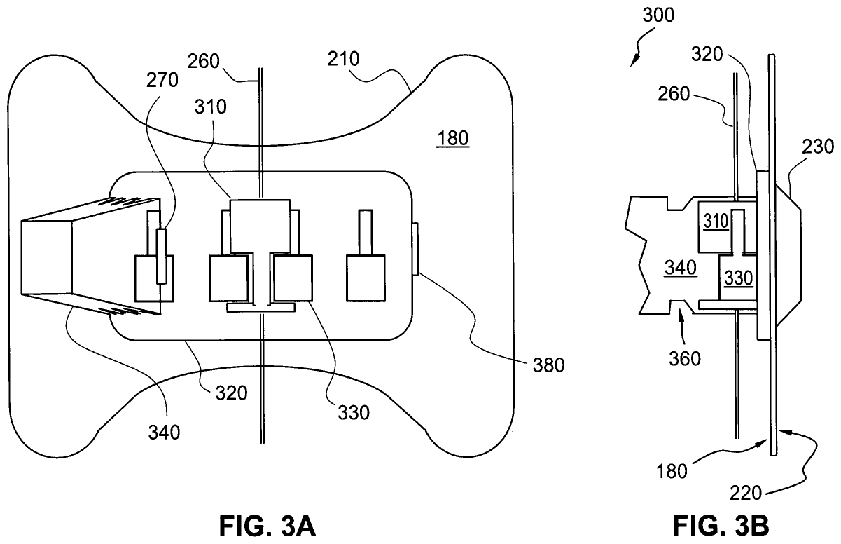

FIG. 2 a shows a plan view 200 of an exemplary embodiment of the stabilizer 170 with a flexible pad 210 with its reverse side 220 facing forward. The pad 210 is preferably composed of a soft foam sheet to avoid irritation to the patient's torso 130. A folded and adhesive foot 230 attaches to the reverse side 220 for adhering to the patient's skin on the torso 130. The foot 230 can optionally include curved tapering wings to mitigate corner edges.

The foot 230 includes a removable film 240 (e.g., paper) to expose the adhesive upon being peeled off by a pull tab 250. The pad 210 can be available with either a tricot base (breathable fabric) or as a foam sheet with perspiration holes to provide air flow to the skin. The exemplary stabilizer 170 provides a mechanism for disposing tube or wire tether lines 260 in proximity to the torso 130.

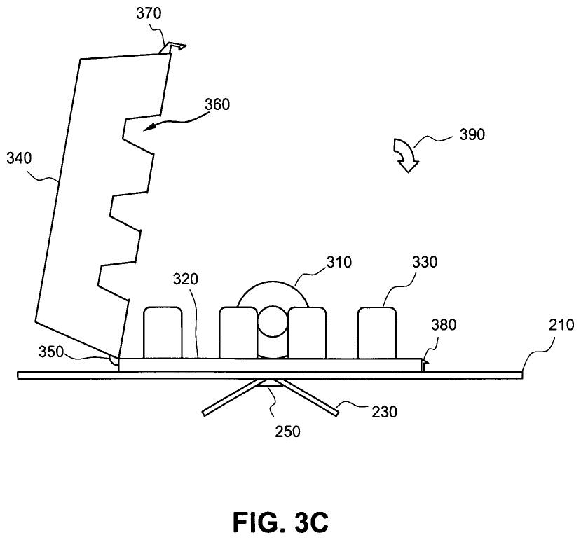

FIG. 3 shows plan and elevation views 300 of the stabilizer 170 for disposing an instrument, such as a transducer 310, aligned to the phlebostatic axis 140 while attaching to the lines 260. The stabilizer 170 includes an instrument holder 320 on the pad's obverse side 180 facing forward in the plan view. Upon disposition on the patient, the holder 320 is centered to the phlebostatic axis 140 and its obverse side faces forward in the plan view.

Transducer clamps 330 are disposed along the holder 320 to be parallel to the midchest line 160 when the pad 210 is disposed on the patient's torso 130. The exemplary clamps 330 constitute flexible stubs between which the transducer 310 squeezes therebetween. A protection cover 340 pivots on the holder 320 along one lateral side by a hinge 350 to swing between open and close positions.

Views 300 shows the protection cover 340 in the open position extending away from the holder 310. The right elevation view illustrates the cover 340 in cutaway. Serration gaps 360 are disposed along the longitudinal edges of the cover 340. The gaps 360 provide cavities for avoiding the lines 260 thereby enabling the cover 340 to close over the holder 320 containing the transducer 310 while avoiding the lines 260 to prevent their crimping.

The cover 340 includes an overhang clevis 370 on the lateral side opposite the hinge 350. The holder 320 includes a latch tang 380 to receive the tang 370 when the cover 340 closes over the holder 320. Pivoting on the hinge 350, the cover 340 can swing inward 390 towards the holder 320 for latching together as a lock 430. The clevis 370 can be pulled outward to release the tang 380 and thereby open the cover 340. Alternative mechanisms to latch the cover 340 to the holder 320, such as a swing hook and knob or by snap buttons, can also be considered without departing from the spirit of the invention.

The holder 320 and cover 340 are preferably composed of rigid polymers, e.g., polyethylene terephthalate, that maintain shape while offering flexibility to open and close the lock 430. The clamps 330 can be composed of a more flexible polymer, such as polyethylene. These material selections are exemplary, and not limiting. The illustrations of the holder 320 with four clamps 330 to secure three instruments, corresponding to three gaps 360 along the longitudinal edges of the cover 340 are merely exemplary and not limiting.

The clamps 330 can secure at least one instrument, such as a transducer 310, that connects to monitors via tube or wire lines 260. As an alternative to flexible stubs, the clamps 330 can be spring-loaded clips or Velcro straps. While the stabilizer 170 remains attached to the patient's torso 130, the transducer 310 maintains the same level as the phlebostatic axis 140.

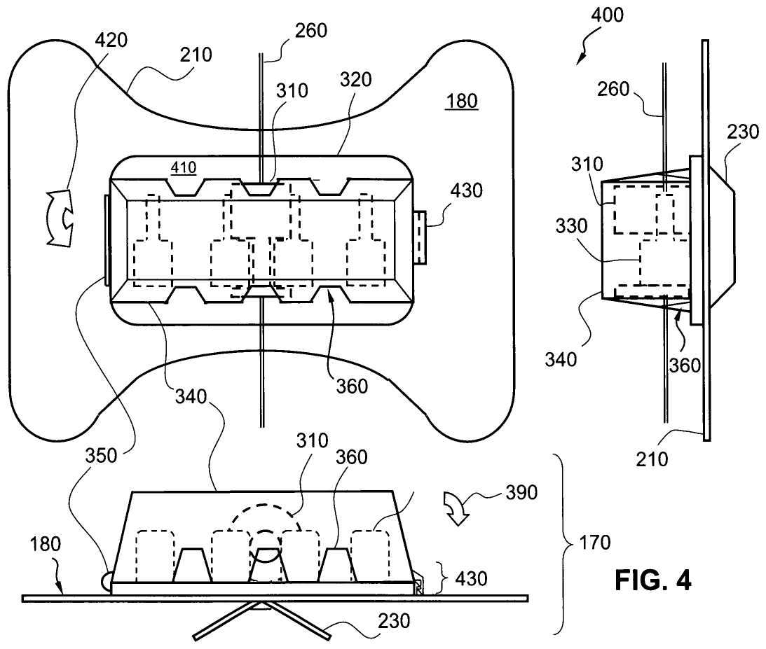

FIG. 4 shows plan and elevation views 400 of the exemplary stabilizer 170 with the cover 340 in the close position over the holder 320 on its obverse side 410. As such, the clamps 330 and transducer 310 are shown in dash line to indicate their obscuration by the cover 340. The instrument holder 320 can swivel on the pad 210 shown by rotation arrows 420, which enables angular freedom to avoid strain or tension on the transducers 310 or its lines 260.

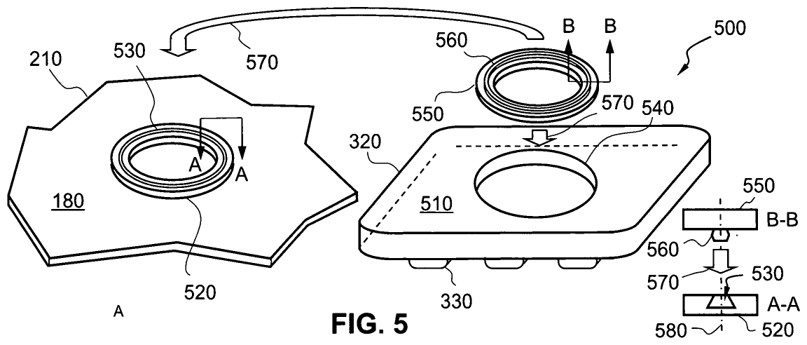

FIG. 5 shows isometric views 500 of a cutaway portion of the pad 210 with its obverse side 180, and the holder 320 with its reverse side 510 facing outward. This reverse side 510 interfaces the obverse side 180 of the pad 210 upon assembly. Note that the clamps 330 mount to the obverse side 410 of the holder 320. The pad 210 includes an indent ring 520 with an annular groove 530 depicted in detail cross-section A-A. The holder 320 includes a cylindrical recess 540 containing a plug ring 550 with an annular race 560 depicted in detail cross-section B-B. The recess 540 can be designed to have a depth to contain both the plug ring 550 and the indent ring 520.

To join the rings to form a swivel joint, the race 560 inserts into the groove 530 in the arrow direction 570 along an annular alignment line 580. Upon this insertion, the plug ring 550 can turn in rotation 420 about their mutual center axes while the indent ring 520 remains stationary. This slip ring configuration enables the pad 210 to remain fixed to the torso 130 while permitting the holder 320 and its instruments to be adjusted in orientation angle independently of the pad's alignment with sufficient friction to inhibit excess spin or backlash. Alternative configurations for the swivel joint can be contemplated to accommodate rotation 420, including coaxial shaft and sleeve or other such slip turners.

Exemplary embodiments are directed to the field of medicine and clinical care in the acute and intensive care unit (ICU) setting. Exemplary embodiments presented provide a convenient and protective securement stabilizer 170 for at least one implantable hemodynamic monitoring (IHM) device, such as pressure transducers 310 designed to be safely attached to the phlebostatic axis 140 (or closest approximate) on the skin of the patient's torso 130. This exemplary stabilizer 170 is projected to be applicable in tight spaces, ambulance transport and specific circumstances where conventional transducer setup can be untenable. The exemplary device is also more convenient in certain patients and may also be substituted for a conventional setup.

In certain acute patient populations, invasive methods of hemodynamic monitoring are required for real time, accurate information regarding a specific patient's hemodynamic status. This is in contrast to indirect methods of hemodynamic monitoring (e.g., external blood pressure cuff pressures). The two commonly used methods of IHM of concern in this context are Pulmonary Artery (PA) and Arterial line (A-line) pressures.

Both monitoring systems operate by being maintained at a level reference point. For both A-lines and PAs, this point is traditionally the phlebostatic axis 140—a point described at the fourth intercostal, mid anterior-posterior surface of the chest—i.e., the upper portion of the torso 130.

The phlebostatic axis 140 describes an area of the surface of the torso 130 that corresponds with the anatomical position of the right atrium. This becomes the reference point for the central Venous pressure (CVP) monitored by the PA catheter. The specific anatomic reference point for the A-line is the aortic root. However, in clinical practice, all pressures measured by the PAs and A-lines are conventionally leveled at a single location, the phlebostatic axis 140.

In conventional clinical usage, transducers 310 are traditionally mounted on a plastic holder and attached to a metal pole beside the patient. A commercial bubble leveler is then used by the clinical practitioner to ensure the mounted transducers are level with the phlebostatic axis 140 of the patient's torso 130. This is generally accomplished by raising or lowering the bed 110, or moving the transducer holder up or down on the pole. Generally the patient and bed 110 are left in this position for monitoring. Any movement that changes the height of the phlebostatic axis 140 (e.g., patient sitting up, raising or lowering the bed 110) requires re-leveling by the clinical practitioner. The trigger mechanisms to flush the lines are also located at the phlebostatic axis 140.

While the conventional setup of a transducer set mounted to a pole next to the patient's bed 110 is generally a common in the specific situation of a sedated nonmoving patient in the setting of an ICU room, circumstances exist in which this arrangement is inconvenient or even untenable. A patient who is awake and moving but requires invasive monitoring represents an example of inconvenience. In contrast to a conventional setup, the exemplary device enables greater freedom of movement for the patient, while obviating constant level adjustments by clinical staff.

Situations untenable for conventional setups include those in which the patient is restrained in a confined enclosure, such as transportation in an ambulance setting, or placed in a cramped space, such as a computed tomography (CT) scanner. In these circumstances, the makeshift solution is to disconnect the transducers 310 and lay or tape the individual transducers 310 at the approximate site of the phlebostatic axis 140. In most cases where the conventional setup is deemed untenable or prohibitively inconvenient, the transducers are placed on the bed 110 or stretcher near the patient's side, with the deviation from the phleobostatic axis 140 being deemed acceptable for a temporary period though not optimal. It is recognized that these makeshift solutions are not optimal to adequately protect the transducers 310 from undesirable and inadvertent manipulation, but constitute expedient circumstances in which conventional setup cannot be performed. Exemplary embodiments for the stabilizer 170 obviate these deficiencies.

Objectives of exemplary embodiments include presenting a stabilizer 170 that can be easily attached to the patient's skin, at the level of the phlebostatic axis 140, and secure the transducers 310 from unwanted manipulation (inadvertent flushing, or unscrewing of the line luer locks). Similar devices have been designed to improve versatility of transducer setup, such as the TruClip holder by Edwards Lifesciences of Irvine California. Exemplary embodiments enable the transducer holder 320 to be disposed on a pole with one hand, leaving the other hand free for other tasks.

For exemplary embodiments, once the transducers 310 are locked into the holder 320, the protection cover 340 can be closed and latched to protect the transducer 310 from inadvertent frictional manipulation. The stabilizer 170 is specifically designed to protect the clamps 330 as well as switches on the transducers 310. Inadvertent switching of pressure flow can cause unwanted backflow of blood into the transducer system and clotting of the lines 260.

Once the cover 340 closes, the transducer 310 can still be flushed with a commonly utilized “snap tab” that inhibits inadvertently pulling. In addition, there is no serious consequence to inadvertently flushing the transducer 310. Further manipulation of the transducer (such as for zeroing) can be accommodated by quickly opening and subsequently closing the cover 340.

While certain features of the embodiments of the invention have been illustrated as described herein, many modifications, substitutions, changes and equivalents will now occur to those skilled in the art. It is, therefore, to be understood that the appended claims are intended to cover all such modifications and changes as fall within the true spirit of the embodiments.

What is claimed is:

1. A stabilization device for attaching an instrument along a phlebostatic axis of a patient's torso, said instrument having a tether to a monitor, said stabilization device comprising:
a flexible pad that contacts the torso along the phlebostatic axis;
an instrument holder disposed on said pad, said holder including a clamp to secure the instrument; and
a protection cover that connects to said holder by a hinge and pivots along said hinge to swing between an open position and a close position, wherein in said close position said cover contains said clamp and the instrument.

2. The stabilization device according to claim 1, further including on said pad, opposite said holder, an adhesive foot with a peelably removable film that exposes said foot upon removal of said film for disposition on the torso.

3. The stabilization device according to claim 1, wherein said pad is composed of a breathable fabric.

4. The stabilization device according to claim 1, wherein said pad is composed of a foam sheet.

5. The stabilization device according to claim 1, wherein said cover latches to said holder in said close position and unlatches in said open position.

6. The stabilization device according to claim 1, wherein said cover includes at least one gap along a periphery to avoid contact with the tether in said close position.

7. The stabilization device according to claim 1, further including a swivel joint between said pad and said holder for angular independence therebetween.
